(19) United States
(12) Patent Application Publication
(10) Pub. No.: US 2008/0039719 A1
(43) Pub. Date: Feb. 14, 2008

(54) Method for determining an optimal trigger time and device for ECG-triggered recording of an object

(76) Inventors: Jan Boese, Eckental (DE); Markus Lendl, Ottensoos (DE)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/789,928

(22) Filed: Apr. 26, 2007

(30) Foreign Application Priority Data
Apr. 27, 2006 (DE) 10 2006 019 692.9

(51) Int. Cl. A61B 6/12; H05G 1/60

(52) U.S. Cl. 600/428; 378/22; 378/95; 600/425

(57) ABSTRACT

There is described a method for determining an optimal trigger time for an ECG-triggered recording of an object. In this respect, a time sequence of dynamic images is firstly acquired with simultaneous recording of an ECG signal, then a time in the cardiac cycle is assigned to each dynamic image, and finally the dynamic images are analyzed, for example by calculation of a degree of similarity, in order to identify the time of minimal movement of the object within the time sequence.

### CROSS REFERENCE TO RELATED APPLICATIONS

This application claims priority of German application No. 10 2006 019 692.9 DE filed Apr. 27, 2006, which is incorporated by reference herein in its entirety.

### FIELD OF INVENTION

The invention relates to a method for determining an optimal trigger time for an ECG-triggered recording of an object in or in the vicinity of the heart with a medical imaging method and also a corresponding device for ECG-triggered recording.

### BACKGROUND OF INVENTION

Diagnostic or surgical operations on the heart are mostly carried out in an image-controlled manner, that is to say that X-ray images, so-called fluoroscopic images, are recorded continuously during the intervention. So-called interventional X-ray systems such as angiography systems or C-beam X-ray devices, for example, are preferably used for this purpose. In the case of the latter, the X-ray tube and X-ray detector are arranged on opposite arms of a C-beam, which can be traversed in any desired angles, so-called angulations, around the patient in order to permit the recording of X-ray images from any desired projection directions.

One such image-controlled operation on the heart is, for example, coronary angioplasty. In this respect, a narrowed coronary artery is expanded with the aid of an interventional inserted balloon. In the case of most coronary operations, a stent is employed in this respect nowadays. This involves an unfolding tubule made of wire mesh, which prevents a re-narrowing of the artery.

An X-ray angiography system is typically employed, as mentioned above, for the purposes of displaying the coronary blood vessels and monitoring the operation. While blood vessels can be displayed very effectively by using angiography following infusion of contrast medium, the depiction of stents is fundamentally more difficult since the very thin wires provide a low X-ray contrast. A reliable depiction is nevertheless needed in order to be able to position stents optimally and in order to be able to assess the state of expansion.

### SUMMARY OF INVENTION

There are various ideas for improving the depiction of stents. Because of the movement of the heart, dynamic X-ray imaging with, typically, 15 or 30 images per second is utilized in order to depict the stent. The basic problem is that the exposure time per image has to be kept short because of the movement of the object and this results, in the presence of an X-ray power limited by the angiography system, in a lower maximal signal-to-noise ratio.

One idea is to calculate an average value image from a plurality of X-ray images recorded one after another in order to improve the signal-to-noise ratio. Because of the movement of the heart, the images nevertheless have to be registered with respect to each other prior to the averaging process, which typically requires user interaction and involves potential errors.

A second method is to record a single image with a higher X-ray dose. In order to achieve the high dose in spite of limitations of the X-ray system, an extended recording time (typically more than 20 milliseconds) is used. Such long recording times normally result in movement blurring because of the movement of the heart. This can be prevented if the image acquisition is effected in an ECG-triggered manner and in fact in such a way that the recording time falls as far as possible into a cardiac phase in which the object under examination moves as little as possible (for example at 80% of the R-R interval). This method has the advantage that no costly or time-consuming image processing, which is susceptible to errors, is needed. Nevertheless, it also has a serious problem: in practice, it is difficult to utilize a fixed trigger time since different segments of the coronary arteries differ in their movement dynamics, and the optimal trigger time is therefore dependent on the position of the stent. Aside from this, the precise time sequence of the cardiac movement is affected by numerous other parameters including, among other things, the local position of the instrument in the heart, the angulation of the X-ray system, the current heart rate, the individual pumping function of the heart, and the individual anatomy.

An object of the present invention is to improve the depiction of stents and similar objects with medical imaging methods in or on the moving heart.

It achieves said object with the characterizing features of the independent claims. Advantageous embodiments of the invention are specified in the dependent claims.

A method according to the invention comprises the following steps: acquisition of a time sequence of dynamic images of the object over at least one cardiac cycle, with simultaneous recording of an ECG signal; assignment of each dynamic image in the time sequence to a time in the cardiac cycle; analysis of the dynamic images for the purposes of identifying a time of minimal movement of the object within the time sequence.

As a result, an optimal trigger time is determined at which the heart is moving as little as possible precisely at the position of the object of interest. Following this, an ECG-triggered recording with higher dose (DR recording) for the purposes of depicting the object, for example a stent, can then be effected at said trigger time.

By particular preference, the invention is employed in the case of X-ray angiography systems, but use in the case of magnetic resonance or ultrasound systems is also conceivable since the same problems exist there in the case of defining a suitable trigger time.

By particular preference, the dynamic images are X-ray images, in particular fluoroscopic recordings or angiographic acquisitions.

The dynamic images are recorded, for example, at a rate of 10 to 60 images/sec, and by particular preference 15 to 30 images/sec.

The object preferably involves an interventional instrument, in particular a stent, stent marker, catheter or guide wire. But the invention can also be used to establish the optimal ECG trigger time for recording a specific anatomical structure of the heart, for example a specific heart valve or a vascular structure.

The method for determining the optimal trigger time operates as follows: firstly a time sequence of dynamic images over at least one cardiac cycle is acquired. Structures with high contrast for the X-ray imaging (for example blood vessels filled with contrast medium) should preferably be present in the image area. Then a time in the cardiac cycle (also referred to as “cardiac phase”) is assigned to each of these dynamic images. This time can, for example, be defined in percent of the average RR cycle′ (0 to 100%), or alternatively as a time interval in milliseconds after the R peak.

Then the dynamic images are analyzed for the purposes of identifying a time of minimal movement of the object. This takes place preferably by the fact that a degree of similarity between two successive images in the time sequence is determined in each case. This results in a sequence of degrees of similarity. The degree of similarity quantifies the extent of agreement between the two images. Suitable degrees of similarity are, for example, the cross-correlation and mutual information. In the case of the cross-correlation, the following term is calculated:

$$M = \frac{cor ( A , B ) \cdot cor ( A , B )}{var ( A ) \cdot var ( B )}$$

(A: first image, B: second image, cor: covariance, var: variance)

Mutual information is a degree of similarity employed frequently in multi-modal image registration, which does not require any linear connection between the images.

The time of minimal movement of the object is then established on the basis of the images with the greatest degree of similarity, since it can be assumed in the case of great similarity that the movement is small.

As defined in an advantageous embodiment only an automatically or manually defined image extract is used in each case for the purposes of analysis or for the purposes of determination of the degrees of similarity between the dynamic images, which extract contains the object or a structure situated in the vicinity of the object. As already mentioned above, the object of interest itself (for example a stent) often only provides low X-ray contrast. It is therefore sensible to carry out the movement analysis, instead of with the object, with the aid of a structure situated in the vicinity of the object with high X-ray contrast, for example a blood vessel filled with contrast medium.

In order that the trigger time is optimal for the area around the object, the degree of similarity is preferably not determined between the complete images, but instead only between image extracts defined automatically or manually in each case. This can take place, for example, by means of collimation on a relatively small image extract. As defined in one embodiment, said image extract lies at a predetermined position, for example in the center of the image, in the case of each image in the time sequence, and preferably has a fixed size. The user then has to move the object into the center prior to the recording of the dynamic images.

As defined in a further advantageous embodiment, the shape and position of the image extract (which may also have an irregular shape in this case) are defined by means of an automatic object recognition on one or more dynamic images. This comes into consideration in particular in the event that the object of interest is readily visible on the dynamic images (for example a stent, a specific segment of a coronary artery or a characteristic bifurcation). Alternatively, the object can also be selected by using interaction by a user, for example by using a cursor. The image extract that must be used for calculating the degree of similarity is then restricted to the object of interest and an environment around it.

A further improvement results if the object of interest is tracked in its movement over the time sequence since then an optimal limitation of the image extract for calculating the degree of similarity can be achieved.

The dynamic images are preferably pre-processed prior to the analysis in order to suppress image components that are not connected to the heart movement. In the case of fluoroscopic images, for example, filters can be used that emphasize the instruments (for example guide wires and stent markers) and suppress the background. Other image processing steps, such as a histogram equalization, for example, are also sensible in order to eliminate influences that are not connected to the heart movement.

The patient table is often moved during the acquisition of fluoroscopic images. In order to compensate for patient table movements of this type during the recording, either the dynamic images can be corrected in accordance with the measured (for example mechanically) table displacement and/or image-based registration methods are employed in order to register the dynamic images in their spatial relationship with respect to each other for the purposes of compensating for the movement of the patient table. In this respect, two-dimensional translation movements between the images are removed prior to calculation of the degree of similarity.

The invention is also directed at a method for ECG-triggered recording of an object in the heart with a medical imaging method. In this respect, an optimal trigger time is firstly determined with the method described above. Then the trigger time found is used for the purposes of recording an ECG-triggered image of the heart. By particular preference, the recording of the ECG-triggered image is effected with an increased X-ray dose. As a result, the recording takes longer than a fluoroscopic recording; but this does not result in movement artifacts due to the optimal trigger time. As a result, objects with low X-ray contrast can also be displayed effectively.

Finally, the invention is also directed at a device for ECG-triggered recording of an object, which includes an imaging device for recording images of the heart, an ECG recording system, a control unit for controlling the recording of the images and also of the ECG signal, and a data store for storing the image data and also the recorded ECG signal. The device is characterized in that the control unit is set up to carry out the aforesaid method for determining an optimal trigger time.

The workflow with said device is preferably improved by the fact that a time sequence of dynamic images is stored continuously in the background for a specific angulation of a C-beam device, which images are then subsequently used for the purposes of analysis in the case of selection of the ECG-triggering. In the event that no images are available for the angulation used, the user is requested to firstly carry out a fluoroscopic recording or a non-ECG-triggered recording.

### BRIEF DESCRIPTION OF THE DRAWINGS

The invention is now described in more detail on the basis of exemplary embodiments with reference to the enclosed drawings.

The drawings show:

FIG. 1 A schematic representation of a device as defined in an exemplary embodiment of the invention;

FIG. 2 A flowchart of a method as defined in an embodiment of the invention; and

FIG. 3 A schematic representation of four dynamic images in a time sequence.

### DETAILED DESCRIPTION OF INVENTION

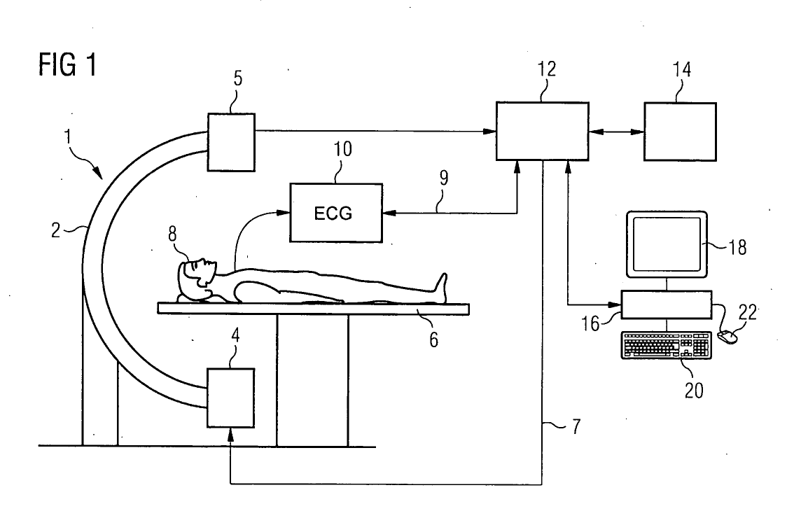

FIG. 1 shows, as an example, a medical imaging device in which the invention can be employed, an angiography system 1. This has a C-beam 2, on the opposite arms of which an X-ray source 4 and a detector 5 are fixed. The C-beam 2 can be traversed around a patient table 6, on which a patient 8 is supported. Furthermore, an ECG system 10 is connected to the patient 8. Systems of this type are known in the prior art and do not need to be explained further here.

The measured image data from the detector 5 and also the ECG signal from the ECG system 10 are transmitted to a control unit 12 in the customary manner. Said control unit controls the image recording and also the recording of the ECG signal, as indicated by the arrows 7 and 9. The control unit 12 is connected to a data store 14 for storing the image data and also the ECG signal. Moreover, the control unit 12 is connected to a user terminal 16. The terminal is, for example, a commercially available PC and is preferably equipped with a monitor 18 and also customary input devices such as a keyboard 20 and a mouse 22 or similar. Alternatively, the control unit 12 and user terminal 16 and also where relevant the data store 14 can be integrated in a single computer.

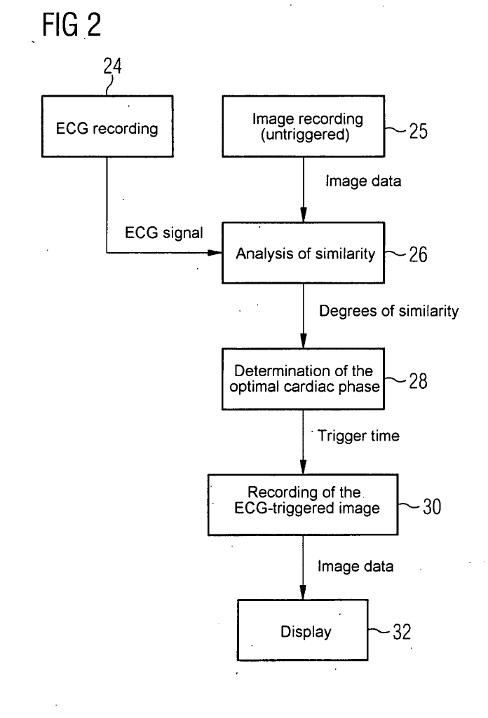

An example of the method according to the invention is now explained on the basis of FIG. 2: in steps 24 and 25, both an ECG signal and also untriggered image data are simultaneously recorded. The dynamic images are subjected to an analysis of similarity in step 26. The degrees of similarity established, for example by means of cross-correlation, in this respect are used in step 28 to determine the cardiac phase with minimal movement of the object of interest. It is assumed in this respect that in the case of great similarity of images recorded one after another, the movement of the object of interest is minimal.

The trigger time established in this way is used in step 30 for recording an ECG-triggered image. This can be an X-ray image with an increased dose. Alternatively or additionally, ECG-triggered recordings of a plurality of cardiac cycles can also be averaged in order to further improve the signal-to-noise ratio and to permit the display of low-contrast objects also.

In step 32, the data of the ECG-triggered image is, for example, transmitted to the user terminal 16 and displayed on the screen 18.

Steps 24 to 30 are controlled or carried out by the control unit 12.

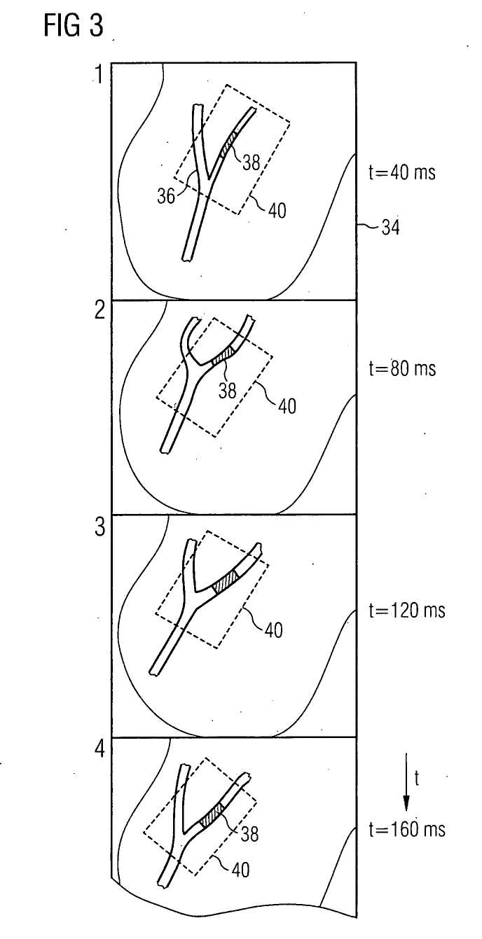

An example of the selection of an image extract on the dynamic images is represented in FIG. 3. Here, four images are represented, by way of example, out of a time sequence of dynamic images 34 that were recorded at the times t=40 ms, 80 ms, 120 ms, and 160 ms after the R peak.

A vascular bifurcation 36 is represented schematically in the images. A stent 38 is situated in one of the vascular branches.

The degrees of similarity between the respectively successive dynamic images 34 is not determined for the overall images in this example, but instead just for the image area within the image extract 40 marked by a broken line. In the example shown, said image extract is rectangular and remains constant over the time sequence, while the section of blood vessel moves slightly through the image extract—for example between images 1 and 2. In other embodiments, in particular if the movement of the stent is so great over the cardiac cycle that the stent partly moves out of the image extract, the image extract 40 can also be shifted over the time sequence in such a way that it follows the position of the stent, or can also be adapted in its shape and/or size.

The image extract 40 can be defined manually by a user on the first image, for example. Alternatively, an automatic object recognition can also be carried out in order to identify the bifurcation and define the image extract correspondingly. It is important in this respect that the degree of similarity is not distorted by the fact that the image extract moves together with the object of interest and a greater similarity than is actually present is calculated as a result.

In the example shown in FIG. 3, it can be seen, for example, that the stent 38 clearly moves through the image extract 40 between images 1 and 2 and also turns. Between images 3 and 4, on the other hand, the stent remains almost constant so that a great degree of similarity and therefore a low object movement is found here. The optimal trigger time established could therefore lie somewhere between 120 ms and 160 ms, while the degrees of similarity between the other images can also be taken into account here for the purposes of establishing the precise optimal trigger time by means of interpolation or similar.

The invention permits an automatic determination of the optimal trigger time and thus makes ECG-triggered recording viable, in particular for cardiological surgery. With the aid of this technique, interventional tools such as stents can be depicted better with X-rays, which can improve the quality and safety of cardiological operations.

1.-19. (canceled)

20. A method for determining a trigger time for an ECG-triggered recording of an object in or in a vicinity of a heart in a body based upon a medical imaging method, comprising:
acquiring a time sequence of dynamic images of the object over at least one cardiac cycle;
recording an ECG signal simultaneously to the acquiring of the images;
assigning the images in the time sequence to a time in the cardiac cycle; and
identifying a time of minimal movement of the object within the time sequence based upon the images.

21. The method as claimed in claim 20,
wherein a degree of similarity between two successive images in the time sequence is determined, and
wherein the images with the greatest degree of similarity are assigned to the time of minimal movement of the object.

22. The method as claimed in claim 21, wherein the degree of similarity is determined based upon a cross-correlation or a mutual information.

23. The method as claimed in claim 20, wherein the object is an interventional instrument.

24. The method as claimed in claim 23, wherein the interventional instrument is selected from the group consisting of: a stent, a stent marker, a catheter, and a guide wire.

25. The method as claimed in claim 20, wherein the images are X-ray images.

26. The method as claimed in claim 20, wherein the time of minimal movement of the object within the time sequence is identified based upon an image extract, wherein the extract contains the object or a structure situated in the vicinity of the object.

27. The method as claimed in claim 26, wherein the image extract is at a predetermined position of the images in the time sequence.

28. The method as claimed in claim 26, wherein the image extract follows the movement of the object or the structure on the dynamic images over the time sequence.

29. The method as claimed in claim 26, wherein a shape of the image extract is defined based upon an automatic object recognition on one or more images.

30. The method as claimed in claim 26, wherein a position of the image extract is defined based upon an automatic object recognition on one or more images.

31. The method as claimed in claim 20, wherein the images are pre-processed prior to the time of minimal movement of the object within the time sequence being identified in order to suppress image components that are without connection to a heart movement.

32. The method as claimed in claim 20, wherein the dynamic images are registered in spatial relationship with respect to a movement of a patient table moved during the acquisition.

33. A method for ECG-triggered recording of an object in or in a vicinity of a heart based upon a medical imaging method, comprising:
determining a trigger time by:
acquiring a time sequence of dynamic images of the object over at least one cardiac cycle,
recording an ECG signal simultaneously to the acquiring,
assigning the images in the time sequence to a time in the cardiac cycle, and
identifying a time of minimal movement of the object within the time sequence based upon the images; and
recording an ECG-triggered image of the heart based upon the trigger time.

34. The method as claimed in claim 33,
wherein the medical imaging method is an X-ray method, and
wherein the recording of the ECG-triggered image is recorded with an increased dose compared to an dose used for determining the trigger time.

35. The method as claimed in claim 33, wherein the medical imaging method is an X-ray method, and the ECG-triggered image is generated based upon an averaging of a plurality of ECG-triggered recordings of a plurality of cardiac cycles.

36. A device for an ECG-triggered recording of an object in a heart, comprising:
an imaging device that records an image of the heart;
an ECG recording system;
a control unit that:
controls the recording of a plurality of images and of the ECG signal,
acquires a time sequence of the images of the heart over at least one cardiac cycle, with simultaneous recording of an ECG signal,
assigns a time in the cardiac cycle to each dynamic image in a time sequence, and
analyzes the dynamic images for the purposes of identifying a time of minimal movement of the object within the time sequence; and
a data storage to store the image data and the recorded ECG signals.

37. The device as claimed in claim 36, wherein the images are recorded at a rate of 15 to 30 images/sec.

38. The device as claimed in claim 36, wherein the imaging device is a C-arm X-ray device and the data storage stores images over a cardiac cycle at an angulation of the C-arm and makes the images available for subsequent determinations of an optimal trigger time at that angulation.

39. The device as claimed in claim 36, wherein the object is an interventional instrument selected from the group consisting of: a stent, a stent marker, a catheter, and guide wire.
